(19) United States
(12) Patent Application Publication
(10) Pub. No.: US 2011/0059982 A1
(43) Pub. Date: Mar. 10, 2011

(54) AZAINDOLE COMPOUNDS FOR TREATMENT OF CENTRAL NERVOUS SYSTEM DISORDERS

(75) Inventors: Timo Heinrich, Gross-Umstadt (DE); Michael Katzer, Egelsbach (DE)

(73) Assignee: MERCK PATENT GESELLSCHAFT MIT BESCHRANKTER HAFTUNG, Darmstadt (DE)

(21) Appl. No.: 12/922,567

(22) PCT Filed: Feb. 14, 2009
(86) PCT No.: PCT/EP09/01055

§ 371 Date: Sep. 14, 2010

(30) Foreign Application Priority Data
Mar. 14, 2008 (EP) 08004860.6

(51) Int. Cl. A61K 31/496; C07D 471/04; A61K 31/4545; A61P 9/12; A61P 25/28; A61P 25/22; A61P 25/24; A61P 25/14; A61P 25/18

(52) U.S. Cl. 514/253.04; 544/362; 546/113; 514/300

(57) ABSTRACT

Azaindole derivative compounds are described. The compounds have an optionally substituted azaindole core linked to a carbocyclic ring having at least one nitrogen atom and further bound to an optionally substituted aryl ring. A process for preparing these compounds, compositions comprising them, and methods of using them to treat disorders of the central nervous system are described.

### FIELD OF THE INVENTION

This application is in the field of pharmaceutical compounds for the treatment of central nervous system disorders.

### BACKGROUND OF THE INVENTION

Compounds that block serotonin and 5-HT_{1A} receptors and inhibit serotonin reuptake have been known and used for decades to treat disease states such as depression, anxiety and epilepsy. In addition to their serotonin agonist and antagonist activities, these molecules also were found to block the binding of serotonin ligands to hippocampal receptors (Cossery et al., Eur. J. Pharmacol. 1987, 140:143), and to modify the accumulation of DOPA in the corpus striatum and the accumulation of 5-HT in the nuclei raphes (Seyfried et al., Eur. J. Pharmacol. 1989, 160:31-41). Administration of such compounds has resulted in the lowering of blood pressure in catheterized, conscious, hypertensive rats (strain: SHR/Okamoto/NIH-MO-CHB-Kisslegg; method: q.v. Weeks and Jones, Proc. Soc. Exptl. Biol. Med. 1960, 104:646-648). Thus, the same compounds find utility both as analgesics and hypotensives. Moreover, these compounds also are useful in the prophylaxis and control of sequelae of cerebral infarctions like stroke and cerebral ischemia.

Anxiety and depression are the most important of all therapeutic indications related to 5-HT disorders since they affect nearly 350 million people worldwide according to the World Health Organization (WHO statistics, 2007). Historically, first generation drugs that enhanced serotonin neurotransmission were non-selective and exhibited undesireable side-effects. In the 1980s, serotonin selective reuptake inhibitors (SSRIs) proved to have fewer side effects than their earlier counterparts, but still stimulated serotonergic sites that slowed the onset of desired drug action. For example, benzoxazine and phthalimide derivative compounds were used for the treatment of CNS disorders like depression, schizophrenia, anxiolytic and antihypertensive disorders by Merrell Dow Pharmaceuticals, Inc. (WO 89/07596; U.S. Pat. No. 4,612,312), and optionally substituted indolepyridyl derivative compounds for this purpose were taught by Malleron et al. (Malleron et al. J. Med. Chem., 1983, 36:1194-1202). However, these SSRIs were effective in less than two-thirds of the patients who received them. Delayed onset of drug action was believed attributable to the increased concentration of serotonin in or near serotonergic cell bodies that activate 5-HT_{1A} autoreceptors, which in turn resulted in decreased cell firing activity and decreased serotonin release in major forebrain areas. This is a naturally-occurring negative feedback mechanism that limits the amount of serotonin located within a synapse that is ready for release. With time, the 5-HT_{1A} autoreceptors become desensitized, and only then are SSRIs permitted their full expression of antidepressant drug activity (Perez et al., Lancet 1997, 349:1594-97).

LePaul et al. suggested that 5-HT_{1A} autoreceptors suppressed the firing of 5-HT neurons, thereby dampening the desired therapeutic effect (LePaul et al., Arch. Pharmacol. 1995, 352:141). Subsequent studies by the same authors showed that after a number of weeks, administration of serotonin reuptake inhibitors resulted in the desensitization of 5-HT_{1A} autoreceptors, which then permitted full antidepressant effects. Thus, overriding the negative feedback effect of 5-HT_{1A} autoreceptors antagonists held the promise of increasing and accelerating clinical antidepressant effects.

Compared to SSRIs, 5-HT_{1A} autoreceptor agonists or partial agonists act directly on postsynaptic serotonin receptors to increase serotonin neurotransmission during the SSRI latency effect period. Feiger and Wilcox demonstrated that buspirone and gepirone were clinically effective 5-HT_{1A} partial agonists (Feiger, A., Psychopharmacol. Bull. 1996, 32:659-65). The addition of buspirone to standard SSRI treatment produced a marked improvement in patients previously unresponsive to standard treatment for depression (Dimitriou, E. J. Clin. Psychopharmacol. 1998, 18:465-9).

In 1996, Artigas et al. suggested that a molecule that combined the effects of overriding 5-HT_{1A} autoreceptor activity and inhibiting 5-HT receptor activity could provide a robust, fast-acting antidepressant effect (Artigas et al., Trends in Neuroscience 1996,19:378-383). Compounds used for the treatment of depression and anxiety included azaindoles and optionally substituted pyrrolopyridine derivatives in particular (American Home Products, Inc., WO 00/64898); aryl piperazinyl cyclohexyl derivative compounds (Eli Lilly Co., EP 0 714894A1; U.S. Pat. No. 5,627,196); and benzofuranyl- and benzothienyl-piperazinyl quinoline analogs (Wyeth, U.S. Pat. No. 7,276,603). To date, however, no marketed drug exists that successfully exhibits a dual mode of action as a 5-HT reuptake inhibitor and a 5-HT_{1A} receptor agonist. Only Vilazodone from Clinical Data, Inc., now in Phase III clinical trials at the U.S. F.D.A. for the treatment of depression, holds the promise of having such activity, but metabolites of Vilazodone have been discovered that suggest the compound is subject to enzymatic degradation (Hewitt et al., Drug Metab. Dispos. 2001, 29:1042; Heinrich et al., Bioorg. Med. Chem. Lett. 2004, 14(10):2681-84).

Thus, there remains a need to provide a single agent having a dual mechanism of action that will block or inhibit serotonin reuptake while simultaneously acting as an agonist at 5-HT_{1A} receptors to permit serotonin release.

There also exists a need to provide a single agent that exhibits desired activity without undesirable side effects on 5-HT and 5-HT_{1A} receptors in the central nervous system for treating diseases such as depression and anxiety.

### SUMMARY OF THE INVENTION

The present invention provides novel compounds that have a dual capacity for affecting serotonin levels in the central nervous system (CNS), and so are useful in the prevention and treatment of a variety of diseases and/or conditions including anxiety, depression, psychoses, neuroses, stroke, hypertension, Parkinson's Disease, Alzheimer's syndrome, Tourette's syndrome, Huntington's Chorea, Lewy body dementia, and generalized pain. The invention also provides synthetic processes for making the compounds of the invention, and pharmaceutical compositions containing these compounds. Further, methods of using the compounds for the prevention and treatment of certain CNS disorders are included herein.

In a first aspect, the present invention provides a compound having a structure according to Formula (I):

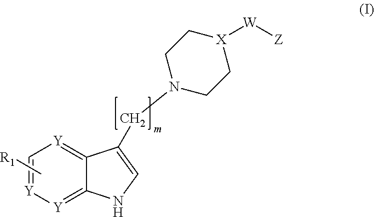

wherein
X is N or CH;
Y each independently is N or CH;
W is (CH₂)ₙ, O, S or N;
R₁ is OH, OA, CN, halo, COR₃, CH₂R₃, or SO₂R₃;
R₃ is OH, OA, NH₂, NHA, or NA₂;
A is C₁₋₆ alkyl that optionally may be substituted or unsubstituted;
Z is a 3-12 membered, unsaturated or unsaturated, mono- or polycyclic ring optionally having one to four heteroatoms and that may be substituted or unsubstituted;
m is 2-6;
n is 0-4;
   or a racemic mixture, enantiomer, diastereoisomer, physiologically acceptable salt, solvate, hydrate, solution or prodrug thereof.

In a preferred embodiment, the compound according to Formula I is incorporated into a pharmaceutical formulation along with one or more pharmaceutically acceptable diluent, excipient, carrier, etc. Those of skill in the art will recognize the overlap in the terms “diluent”, “excipient” and “carrier”.

In a further aspect the invention provides a method for treating or preventing a disease or condition that is a member selected from a neurological disorder, pain, depression, anxiety, dementias and other CNS-related disorders. The method includes administering to a subject in need thereof a therapeutically effective amount of a compound of Formula (I) or a pharmaceutically acceptable salt or solvate thereof.

In yet another aspect the invention provides a kit comprising separate packs of a therapeutically effective amount of a pharmaceutical composition that comprises the compound of Formula I and a pharmaceutically acceptable diluent, excipient or carrier.

### DETAILED DESCRIPTION OF THE INVENTION

### I. Introduction

The present invention provides novel compounds that have a dual capacity for affecting serotonin levels in the central nervous system (CNS) by their antagonistic blocking or inhibitory action on 5-HT receptors and agonist activity on 5-HT_{1A} receptors that permits serotonin release. These compounds have utility in the prevention and treatment of a variety of neurologic disorders and/or conditions. For example, the invention provides methods for treating or preventing anxiety, depression, psychoses, neuroses, stroke, hypertension, neurodegenerative diseases such as Parkinson's Disease and Lewy body dementia, Alzheimer's syndrome, Tourette's syndrome, Huntington's Chorea, and generalized pain. The invention also provides synthetic processes for making the compounds of the invention, and pharmaceutical compositions containing these compounds.

In one aspect the present invention provides a compound having a structure according to Formula (I):

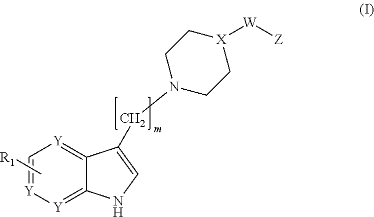

wherein
X is N or CH;
Y each independently is N or CH;
W is (CH₂)ₙ, O, S or N;
R₁ is OH, OA, CN, halo, COR₃, CH₂R₃, or SO₂R₃;
R₃ is OH, OA, NH₂, NHA, or NA₂;
A is C₁₋₆ alkyl that optionally may be substituted or unsubstituted;
Z is a 3-12 membered, unsaturated or unsaturated, mono- or polycyclic ring optionally having one to four heteroatoms and that may be substituted or unsubstituted;
m is 2-6;
n is 0-4;
   or a racemic mixture, enantiomer, diastereoisomer, physiologically acceptable salt, solvate, hydrate, solution or prodrug thereof.

In a preferred embodiment, the compound according to Formula I is incorporated into a pharmaceutical formulation along with one or more pharmaceutically acceptable diluent, excipient, carrier, etc.

In another aspect the invention provides a method for treating or preventing a disease or condition that is a member selected from a neurological disorder, pain, depression, anxiety, dementias and other CNS-related disorders. The method includes administering to a subject in need thereof a therapeutically effective amount of a compound of Formula (I) or a pharmaceutically acceptable salt or solvate thereof.

In yet another aspect the invention provides a kit comprising separate packs of a therapeutically effective amount of a pharmaceutical composition that comprises the compound of Formula I and a pharmaceutically acceptable diluent, excipient or carrier.

### II. Definitions

Where substituent groups are specified by their conventional chemical formulae, written from left to right, they optionally encompass substituents resulting from writing the structure from right to left, e.g., —CH₂O— optionally also recites —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and optionally may be substituted. It can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, optionally includes those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. A “cycloalkyl” or “heterocycloalkyl” substituent may be attached to the remainder of the molecule directly or through a linker, wherein the linker is preferably alkyl. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, difluoromethyl, fluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 7-azaindole, 1-isoquinolyl, 5-isoquinolyl, 2-quinoxalinyl, 5-quinoxalinyl, 3-quinolyl, 6-quinolyl, 1-piperidinyl, 3-benzofuranyl, and 4-benzodioxinyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) optionally includes both aryl and heteroaryl rings as defined above. Thus, the term “arylalkyl” optionally includes those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) optionally include both substituted and unsubstituted forms of the indicated radical. Preferred substituents for each type of radical are provided below.

Substituents for the alkyl and heteroalkyl radicals (including those groups often referred to as alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, and —R₁, wherein R₁ is —OH, O-alkyl, —CN, -halo, —C(O)OH, —C(O)O(alkyl), —C(O)NH₂, —C(O)NH(alkyl), —C(O)N(alkyl)₂, —CH₂OH, —CH₂O(alkyl), —CH₂NH₂, —CH₂NH(alkyl), —CH-₂N(alkyl)₂, —SO₂OH, —SO₂O(alkyl), —SO₂NH₂, —SO₂NH(alkyl), and —SO₂N(alkyl)₂. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted aryl, substituted or unsubstituted heteroaryl, substituted or unsubstituted heterocycloalkyl, —OH, —O-alkyl, —CN, -halo, —C(O)OH, —C(O)O(alkyl), —C(O)NH₂,
—C(O)NH(alkyl), —C(O)N(alkyl)₂, —CH₂OH, —CH₂O(alkyl), —CH₂NH₂, —CH₂NH(alkyl),
—CH₂N(alkyl)₂, —SO₂OH, —SO₂O(alkyl), —SO₂NH₂, —SO₂NH(alkyl), and —SO₂N(alkyl)₂.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S) and silicon (Si) and boron (B).

The phrase “therapeutically effective amount” as used herein means that amount of a compound, material, or composition comprising a compound of the present invention which is effective for producing some desired therapeutic effect by simultaneous blocking or inhibiting of 5-HT reuptake receptors and triggering an agonist-like effect on 5-HT_{1A} autoreceptors in a mammal, thereby blocking the biological consequences of that pathway in the treated cells, at a reasonable benefit/risk ratio applicable to any medical treatment.

The term “pharmaceutically acceptable salts” includes salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., J. Pharma. Science 1977, 66: 1-19). Certain specific compounds of the present invention contain both basic and acidic functionalities that allow the compounds to be converted into either base or acid addition salts.

As used herein, the abbreviation “THF” means tetrahydrofuran; “TBAF” means tetra-n-butyl ammonium fluoride; “DMF” means dimethyl formamide; and “NMP” means N-methylpyrrolidinone.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. For instance, prodrugs for carboxylic acid analogs of the invention include a variety of esters. In an exemplary embodiment, the pharmaceutical compositions of the invention include a carboxylic acid ester. In another exemplary embodiment, the prodrug is suitable for treatment /prevention of those diseases and conditions that require the drug molecule to cross the blood brain barrier. In a preferred embodiment, the prodrug enters the brain, where it is converted into the active form of the drug molecule. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are of use in the methods contemplated by the present invention and are intended to be within the scope of the present invention. “Compound or a pharmaceutically acceptable salt, hydrate, polymorph or solvate of a compound” intends the inclusive meaning of “or”, in that materials meeting more than one of the stated criteria are included, e.g., a material that is both a salt and a solvate is encompassed.

Certain compounds of the present invention possess asymmetric carbon atoms (optical centers) or double bonds; the racemates, diastereomers, geometric isomers and individual isomers are encompassed within the scope of the present invention. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are included.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

In the context of the present invention, compounds that are considered to possess activity as 5-HT receptor antagonists (i.e., blockers/inhibitors) and 5-HT_{1A} receptor agonists are those displaying 50% inhibition of the activity of 5-HT (IC₅₀) at a concentration of not higher than about 500 nM/L, preferably, not higher than about 100 nM/L.

The term “neurological disorder” refers to any condition of the central or peripheral nervous system of a mammal. The term “neurological disorder” includes neurodegenerative diseases (e.g., Alzheimer's disease, Parkinson's disease, Lewy body dementia, and amyotrophic lateral sclerosis), and neuropsychiatric diseases (e.g. Schizophrenia and anxieties, such as general anxiety disorder). Exemplary neurological disorders include Huntington's disease, multi-infarct dementia, viral infection induced neurodegeneration (e.g. AIDS, encephalopathies), psychoses, depression (e.g., bipolar disorder), dementias, and movement disorders such as Tourette's syndrome, stroke, and the like. “Neurological disorder” also includes any condition associated with the disorder. For instance, a method of treating a neurodegenerative disorder includes methods of treating loss of memory and/or loss of cognition associated with a neurodegenerative disorder. Such method would also include treating or preventing loss of neuronal function characteristic of neurodegenerative disorder.

### III. Compositions

### A. Fused Heterocycles

The heterocyclic antagonists/agonists of the invention are characterized by a core-moiety comprising a 4-, 6- or 7-azaindole. In an exemplary embodiment, the core-moiety includes an azaindole heterocyclic ring system that further is substituted at the 3-position on the azaindole core by a ligand-bound chain containing at least one additional heterocyclic moiety. Exemplary heterocyclic moieties include rings, such as piperazinyl, piperidinyl, benzodioxolinyl, furanyl, benzofuranyl, oxazolyl, isoxazolyl, thiazolyl, isothiazolyl, imidazolyl and pyrazolyl moieties, and preferably piperazinyl, piperidinyl, and benzodioxolinyl heterocyclic groups.

In a first aspect, the present invention provides a compound having a structure according to Formula (I):

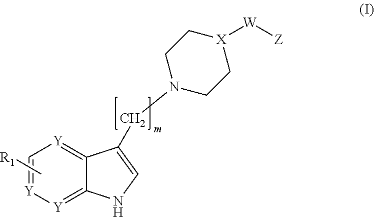

wherein
X is N or CH;
Y each independently is N or CH;
W is (CH₂)ₙ, O, S or N;
R₁ is OH, OA, CN, halo, COR₃, CH₂R₃, or SO₂R₃;
R₃ is OH, OA, NH₂, NHA, or NA₂;
A is C₁₋₆ alkyl that optionally may be substituted or unsubstituted;
Z is a 3-12 membered, unsaturated or unsaturated, mono- or polycyclic ring optionally having one to four heteroatoms and that may be substituted or unsubstituted;
m is 2-6;
n is 0-4;
   or a racemic mixture, enantiomer, diastereoisomer, physiologically acceptable salt, solvate, hydrate, solution or prodrug thereof.

In a preferred embodiment, the compound according to Formula I is incorporated into a pharmaceutical formulation along with one or more a pharmaceutically acceptable diluent, excipient, carrier, etc.

### B. Synthesis of Fused Pyridinyl Pyrrole Analogs

The Larock indole synthesis is the key step in preparing compounds of the present invention (Larock et al., J. Org. Chem. 1998, 63:7652). In this procedure, an indole is synthesized from an ortho-iodoaniline and a disubstituted alkyne, which process is given generally as:

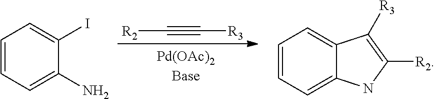

It is understood that the ortho-iodoaniline may be replaced by 2-amino-3-iodo pyridine or another starting material of choice as known to one of skill in the art. Further, it is understood that preparation of an iodinated reactant compound may be accomplished by applying several different methodologies and reagents as known to those skilled in the art. Different solvents and reaction conditions also may be utilized, as known to those skilled in the art, in order to obtain the desired iodinated product.

Reaction of the iodinated compound species with excess disubstituted alkyne in the presence of a base produces the desired substituted indole product. Again, it is understood that any base that is able to support the catalytic transformation can be used, including inorganic as well as organic bases like triethylamine or sodium carbonate. Where R₂ is H and will be transferred into a silyl-group, then a base whose corresponding pKa is greater than 25 is required. Suitable bases include lithium diisopropylamide, sodium or potassium n-butyl-lithium hydride, or sodium or potassium hexamethyl disilane, as examples.

A phenylpiperazinyl- or phenylpyridinyl-containing substituent may be prepared by a number of methodologies known to those of skill in the art, two of which are the nucleophilic substitution reactions exemplified as:

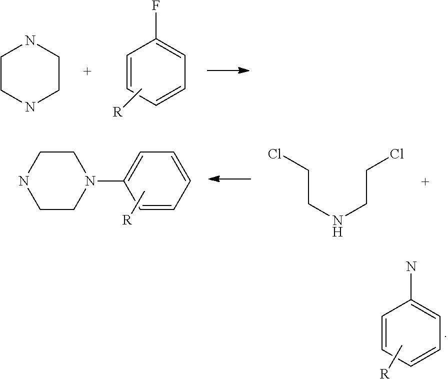

The first reaction of piperazine with a substituted fluoro-benzene was carried out in aqueous solution and heat (the presence of a catalyst is not mandatory). Likewise, the alternative reaction of a bis(2-chloroethyl)amine with a substituted aniline was performed in basic aqueous solution (the presence of a catalyst is not necessary), but may be accomplished as a one-pot microwave-assisted synthesis (see Heinrich et al., J. Med. Chem. 2004, 47(19):4684-92; and Ju and Varma, J. Org. Chem. 2006, 71:135-141). A typical base (catalyst) for these reactions is K₂CO₃, but any catalyst that provides the desired product may be used. Different solvents, and reaction conditions also may be utilized, as known to those skilled in the art. Typical additives utilized in these reactions include halogens, and Iodide, Chloride, and Fluoride in particular, NMP and DMF, as examples.

Finally, addition of a phenylpiperazinyl- or phenylpiperidinyl-containing substituent onto the azaindole scaffold was accomplished by use of a trimethyl silanyl hexynyl analog of the substituent. The silanyl analog was reacted with nicotinonitrile and a phosphenoferrocene dichloro Palladium (II) dichloromethane adduct in a solvent, extracted and purified to obtain the final product. Alternatively, a 5-cyano-3-iodobutylindole may be reacted with the phenylpiperazinyl- or phenylpiperidinyl-containing substituent in the presence of K₂CO₃ and NMP to afford the final product. Where this final product is an ester, the latter can be reacted with K₂CO₃ in methanol, activated by the Mukaiyama reagent, and treated with gaseous ammonia to provide the analogous carboxamide (Heinrich et al., J. Med. Chem. 2004, 47(19):4684-92). Solvents and alternative bases include but are not limited to DMF, NMP, cesium carbonate, and ethyldiisopropylamine.

The molecular weight (m+H⁺) of the product was determined with the aid of electron spray ionization mass spectroscopy. Mass-spectroscopic data were derived from HPLC/MSC runs (HPLC coupled with an electrospray ionization mass spectrometer). The numerical values are, as customary in this procedure, not the molecular weight of the unmodified compounds, but the molecular weights of the protonated compounds. The method is described in Yamashita et al., J. Phys. Chem. 1984, 88:4451-59; and Fenn et al, Science 1989, 246, 64-71.

The purification of the products were achieved by preparative HPLC if not otherwise stated, and given as:
Column: RP 18 (15 μm) Lichresorb 250×50
solvent: A: 98 H₂O, 2 CH₃CN, 0.1% TFA or formic acid
B: 10 H₂O, 90 CH₃CN, 0.1% TFA or formic acid
UV: 225 nm, one range
Flow rate 10 ml/min

The entire synthesis provided herein represents a best but not the only method of preparing a compound of the present invention. It is understood that the present process is not limited to the reagents and conditions given herein, but may be done by any other means known to one skilled in the art that produces the desired product.

4-, 6- & 7-azaindole analogs of the present invention were prepared according to the processes exemplified herein.

### C. Pharmaceutical Compositions

While compounds of the present invention can be administered as the raw chemical, it is preferable to present them as a pharmaceutical composition. According to a further aspect, the present invention provides a pharmaceutical composition comprising a compound of Formula (I) or a pharmaceutically acceptable salt, hydrate or solvate thereof, together with one or more pharmaceutical carrier and optionally one or more other therapeutic ingredients. The carrier(s) are “acceptable” in the sense of being compatible with the other ingredients of the formulation and not deleterious to the recipient thereof. The term “pharmaceutically acceptable carrier” includes vehicles, diluents, excipients and other elements appropriate for incorporation into a pharmaceutical formulation.

The formulations include those suitable for oral, parenteral (including subcutaneous, intradermal, intramuscular, intravenous, peritoneal and intraarticular), rectal, ionotophoretic, and topical (including dermal, buccal, sublingual and intraocular) administration, as well as those for administration by inhalation. The most suitable route may depend upon the condition and disorder of the recipient. The formulations may conveniently be presented in unit dosage form and may be prepared by any of the methods well known in the art of pharmacy. All methods include the step of bringing into association a compound or a pharmaceutically acceptable salt or solvate thereof (“active ingredient”) with the carrier that constitutes one or more accessory ingredients. In general, the formulations are prepared by uniformly and intimately bringing into association the active ingredient with liquid carriers or finely divided solid carriers or both and then, if necessary, shaping the product into the desired formulation. Oral formulations are well known to those skilled in the art, and general methods for preparing them are found in any standard pharmacy school textbook, for example, Remington: The Science and Practice of Pharmacy., A. R. Gennaro, ed. (1995), the entire disclosure of which is incorporated herein by reference.

Pharmaceutical compositions containing compounds of Formula (I) may be conveniently presented in unit dosage form and prepared by any of the methods well known in the art of pharmacy. Preferred unit dosage formulations are those containing an effective dose, or an appropriate fraction thereof, of the active ingredient, or a pharmaceutically acceptable salt thereof. The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose ranges from about 0.1 mg per day to about 7000 mg per day, preferably about 1 mg per day to about 100 mg per day, and more preferably, about 25 mg per day to about 50 mg per day, in single or divided doses. In some embodiments, the total daily dose may range from about 50 mg to about 500 mg per day, and preferably, about 100 mg to about 500 mg per day. It is further recommended that children, patients over 65 years old, and those with impaired renal or hepatic function, initially receive low doses and that the dosage is titrated based on individual responses and/or blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compressing or molding the compound of Formula (I), optionally using one or more additional ingredient. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein. Oral and parenteral sustained release drug delivery systems are well known to those skilled in the art, and general methods of achieving sustained release of orally or parenterally administered drugs are found, for example, in Remington: The Science and Practice of Pharmacy, A. R. Gennaro, ed., pages 1660-1675 (1995). It should be understood that in addition to the ingredients particularly mentioned above, the formulations of this invention may include other agents conventional in the art having regard to the type of formulation in question, for example those suitable for oral administration may include flavoring agents.

Formulations for parenteral administration include aqueous and non-aqueous sterile injection solutions that may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents, while formulations for oral administration also may include flavoring agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described. Formulations for rectal administration may be presented as a suppository with the usual carriers such as cocoa butter or polyethylene glycol. Formulations for topical administration in the mouth, for example, buccally or sublingually, include lozenges comprising the active ingredient in a flavored basis such as sucrose and acacia or tragacanth, and pastilles comprising the active ingredient in a basis such as gelatin and glycerin or sucrose and acacia.

The pharmaceutically acceptable carrier may take a wide variety of forms, depending on the route desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by standard aqueous or nonaqueous techniques. Oral and parenteral sustained release dosage forms may also be used.

Exemplary formulations, are well known to those skilled in the art, and general methods for preparing them are found in any standard pharmacy school textbook, for example, Remington: The Science and Practice of Pharmacy, A. R. Gennaro, ed., pages 1660-1675 (1995).

One aspect of the present invention contemplates the treatment of the disease/condition with the pharmaceutically active agent that may be sold in kit form. The kit comprises a compound of the present invention contained within a syringe, box, bag, and the like. Typically, the kit comprises directions for the administration of the compound. The kit form is particularly advantageous when different dosage concentrations and/or forms (e.g., oral and parenteral) are sold, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). They generally consist of a sheet of relatively stiff material covered with a foil of a preferably transparent plastic material. During the packaging process recesses are formed in the plastic foil. The recesses have the size and shape of the tablets or capsules to be packed. The tablets or capsules are placed in the recesses and the sheet of relatively stiff material is sealed against the plastic foil at the face of the foil which is opposite from the direction in which the recesses were formed. Particular dosage information normally is stamped onto each blister pack.

In another specific embodiment of the invention, a dispenser designed to dispense the daily doses one at a time in the order of their intended use is provided.

### IV. Methods

### A. Methods for Treatment or Prevention

In a further aspect the invention provides a method for treating or preventing a disease or condition that is a member selected from a neurological disorder such as anxiety, depression, stroke, and hypertension. The method includes administering to a subject in need thereof a therapeutically effective amount of a compound of Formula (I) or a pharmaceutically acceptable salt, hydrate, prodrug or solvate thereof:

Subjects for treatment according to the present invention include humans (patients) and other mammals in need of therapy for the stated condition.

Compounds of the invention possess unique pharmacological characteristics with respect to inhibition of serotonin reuptake and influence the activity of the 5-HT_{1A} receptors in the CNS. Therefore, these compounds are effective in treating conditions and disorders (especially CNS-related disorders), which are modulated by serotonin activity. In one embodiment, compounds of the invention are associated with diminished side effects compared to administration of the current standards of treatment.

Accordingly, the present invention relates to methods for increasing the concentration of serotonin in a mammal. Each of the methods comprises administering to a subject in need thereof a therapeutically effective amount of a compound of the invention, for example those of Formula (I), or a pharmaceutically acceptable salt, hydrate, prodrug or solvate thereof.

Compounds of the invention are typically more selective than known serotonin reuptake inhibitors and 5-HT_{1A} agonists, and demonstrate higher selectivity for serotonin reuptake receptors and 5-HT_{1A} autoreceptors relative to binding of known compounds at the 5-HT and 5-HT_{1A} receptor binding sites. The compounds also exhibit an advantageous profile of activity including good bioavailability. Accordingly, they offer advantages over many art-known methods for treating disorders modulated by serotonin levels.

### V. Conditions and Disorders

In one embodiment, the compounds of the present invention are useful for the treatment of neurological disorders, depression, anxiety, hypertension and stroke. Neurological disorders include neurodegenerative diseases (e.g., Alzheimers disease) and neuropsychiatric disorders (e.g., psychoses).

Disorders treatable with the compounds of the present invention include, but are not limited to, depression, dementias, Tourette's syndrome, Huntington's disease, generalized anxiety disorder, phobic anxiety, psychoses, hypertension, stroke, and side effects resulting from the treatment of these conditions.

Compounds of the present invention also have use in the fields of gynecology and endocrinology for treatment of hypogonadism, premenstrual syndrome, secondary amenorrhea, undesired puerperal lactation, and in cerebral disorders such as migraines, and cerebral ischemia.

The following examples are provided to illustrate selected embodiments of the invention and are not to be construed as limiting its scope.

### Examples

### Example 1

### Synthesis of 5-{4-[4-(5-Cyano-1H-pyrrolo[2,3-b]pyridine-3-yl)-butyl]-piperazin-1-yl}-benzofuran-2-carboxylic acid amide

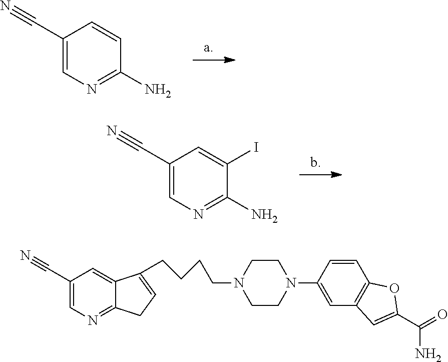 [0111] a.: 67 g of 6-amino-nicotinonitrile were dissolved in 1 L 1,2-dichloroethane, 125 g argentic trifluoro acetate were added and the mixture was refluxed for 7 h. After cooling to room temperature (RT), 143 g of iodine were added. The mixture was heated again for 12 h. Then the temperature was lowered to RT and the salts removed by filtration. The reaction phase was treated with 1 L of water. The aqueous phase was extracted with dichloromethane and the combined organic layers were dried over magnesium sulfate, evaporated and purified by chromatography over silica gel yielding 41 g yellowish crystals 6-amino-5-iodo-nicotinonitrile.
[M+H]⁺: 246
b.: 6 g 6-chloro-1-hexyne were dissolved in 50 ml THF and cooled to −78° C. At that temperature 31 ml of n-BuLi (1.6 M in hexane) were added drop by drop. Within 1 h the reaction mixture was heated to −20° C. At that temperature 50 ml chloro-trimethyl-silane were added and finally the reaction stirred for 12 h at RT. For work up the solution was poured on 50 ml water. After the usual extraction and purification procedure 6.6 g of (6-chloro-hex-1-ynyl)-trimethyl-silane.
[M+H]⁺: 189

6.6 g of (6-chloro-hex-1-ynyl)-trimethyl-silane, 11 g 5-piperazin-1-yl-benzofuran-2-carboxylic acid amide (Reitz et al., J. Med. Chem. 2005, 38:4211-22), and 8.3 mL triethyl-amine were dissolved in 100 mL acetonitrile and heated for 72 h. After cooling to RT the reaction mixture was poured on 100 mL water and 100 mL ethyl acetate. After the usual extraction and purification procedure 3.9 g colorless crystals 5-[4-(6-trimethylsilanyl-hex-5-ynyl)-piperazin-1-yl]-benzofuran-2-carboxylic acid amide were obtained.
HPLC: Chromolite Performance RP18-e 100-4,6 mm
   Gradient: ACN/H₂O with 0.05% formic acid
   Method: Chromolith/Chromolith (extended)
   flow: 3 mL/min
   Retention (Rt): 2.96 min
   [M+H]⁺: 398

1.2 g 5-[4-(6-trimethylsilanyl-hex-5-ynyl)-piperazin-1-yl]-benzofuran-2-carboxylic acid amide, 500 mg 6-amino-5-iodo-nicotinonitrile, 0.1 g lithium chloride, 0.8 g sodium carbonate and 0.1 g 1,1′-bis(diphenyl-phosphino)ferrocenedichloropalladium-(II) dichloromethane adduct were dissolved in 50 ml DMF and heated for 12 h. The black suspension was poured on 50 ml water and extracted with ethyl acetate. After the usual extraction and purification procedure 20 mg of fawn amorphous solid 5-{4-[4-(5-cyano-1H-pyrrolo[2,3-b]pyridine-3-yl)-butyl]-piperazin-1-yl}-benzofuran-2-carboxylic acid amide were obtained.

¹H-NMR (500 MHz, d₆-DMSO) δ 12.18 (br s, 1H), 8.49 (d, 1H, J=1.8 Hz), 8.33 (d, 1H, J=1.8 Hz), 7.99 (br. s, 1H), 7.57 (br. s, 1H), 7.47 (d, 1H, J=9.9 Hz), 7.40 (s, 1H), 7.17 (m, 2H), 6.35 (s, 1H), 3.33 (m, 4H), 3.11 (m, 4H), 2.81 (m, 2H), 2.38 (m, 2H), 1.76 (m, 2H), 1.54 (m, 2H).
HPLC-MS: Chromolite SpeedROD RP-18e 50-4, 6 mm
   solvent A: water+0.1% TFA
   solvent B: acetonitrile+0.1% TFA
   flow: 2.4 mL/min
   gradient: 0,0 min 4% B
      2.6 min 100% B
   Rt: 1.909 min
      [M+H]⁺: 398

### Example 2

### Synthesis of 5-{4-[4-(5-Cyano-1H-pyrrolo[3,2-b]pyridine-3-yl)-butyl]-piperazin-1-yl}-benzofuran-2-carboxylic acid amide

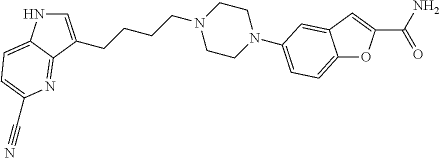

25 g of 5-amino-pyridine-2-carbonitrile were treated as described for 6-amino-nicotinonitrile giving 20 g of fawn crystals 5-amino-6-iodo-pyridine-2-carbonitrile.
HPLC: Chromolite Performance RP18-e 100-4,6 mm
   Gradient: ACN/H₂O witht 0.05% formic acid
   Method: Chromolith/Chromolith (extended)
   flow: 3 mL/min
   Retention (Rt): 1.596 min
      [M+H]⁺: 246

¹H-NMR (500 MHz, d₆-DMSO) δ 7.62 (d, 1H, J=8.3 Hz), 6.97 (d, 1H, J=8.3 Hz), 6.44 (br. s, 2H).

500 mg 5-amino-6-iodo-pyridine-2-carbonitrile and 1.2 g 5-[4-(6-trimethylsilanyl-hex-5-ynyl)-piperazin-1-yl]-benzofuran-2-carboxylic acid amide were treated as described for 5-{4-[4-(5-Cyano-1H-pyrrolo[2,3-b]pyridine-3-yl)-butyl]-piperazin-1-yl}-benzofuran-2-carboxylic acid amide, giving 20 mg of fawn amorphous solid 5-{4-[4-(5-Cyano-1 H-pyrrolo[3,2-b]pyridine-3-yl)-butyl]-piperazin-1-yl}-benzofuran-2-carboxylic acid amide.

¹H-NMR (500 MHz, d₆-DMSO) δ 12.18 (br s, 1H), 7.99 (br.s, 1H), 7.83 (dd, 1H, J=0.8 Hz, J=8.2 Hz), 7.58 (d, 2H, J=8.3 Hz), 7.47(d, 1 H, J=9.3 Hz), 7.40 (s, 1H), 7.17 (m, 2H), 6.47 (s, 1H), 3.31 (m, 4H), 3.10 (m, 4H), 2.79 (m, 2H), 2.37 (m, 2H), 1.77 (m, 2H), 1.56 (m, 2H).
HPLC-MS: Chromolite SpeedROD RP-18e 50-4, 6 mm
   solvent A: water+0.1% TFA
   solvent B: acetonitrile+0.1% TFA
   flow: 2.4 mL/min
   gradient: 0,0 min 4% B
      2.6 min 100% B
   Rt: 1.901 min
      [M+H]⁺: 398

### Example 3

### Synthesis of 3-{4-[4-(2,3-Dihydro-benzo[1,4]dioxin-6-yloxy)-piperidin-1-yl]-butyl}-1H-pyrrolo[2,3-b]pyridine-5-carbonitrile

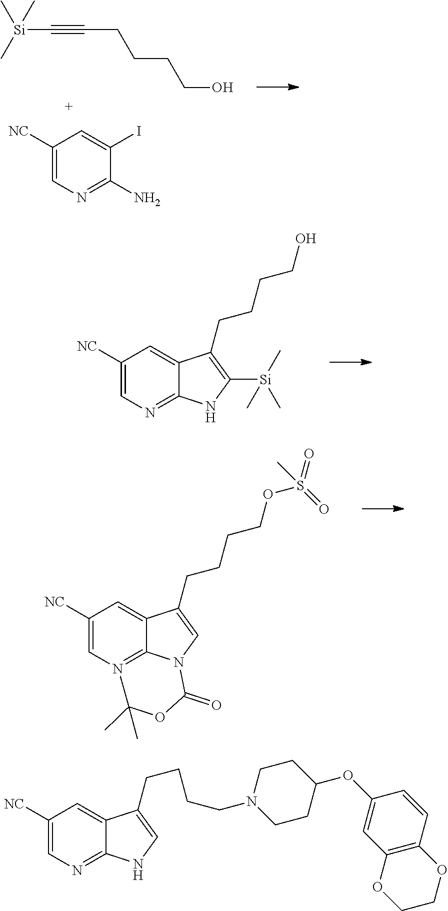

10 g 5-hexyn-1-ol were dissolved in 150 mL THF and cooled to −78° C. 187 ml (1.6 M in n-hexane) buthyl lithium were added drop by drop. After stirring at −20° C. for 1 h 30 ml chloro-trimethyl-silane were added drop by drop at the given temperature. After reacting for 12 h at RT the mixture was worked up with 100 mL water. After the usual extraction and purification procedure 3.3 g of a colorless oil 6-trimethylsilanyl-hex-5-yn-1-ol were obtained.

2.9 g of 6-amino-5-iodo-nicotinonitrile and 2 g 6-trimethylsilanyl-hex-5-yn-1-ol were treated as described for 5-{4-[4-(5-Cyano-1 H-pyrrolo[2,3-b]pyridine-3-yl)-butyl]-piperazin-1-yl}-benzofuran-2-carboxylic acid amid and gave 630 mg brown oil 3-(4-hydroxy-butyl)-2-trimethylsilanyl-1 H-pyrrolo[2,3-b]pyridine-5-carbonitrile.
HPLC: Rt: 2,370 min
HPLC-MS: Rt: 1,429 min

1.8 g 3-(4-hydroxy-butyl)-2-trimethylsilanyl-1 H-pyrrolo[2,3-b]pyridine-5-carbonitrile were dissolved in 50 mL THF and stirred with 9 mL TBAF (1 M in THF)for 12 h at RT. After the usual extraction and purification procedure 520 mg 3-(4-hydroxy-butyl)-1H-pyrrolo[2,3-b]pyridine-5-carbonitrile were obtained.
HPLC: Rt: 2,510 min
HPLC-MS: Rt: 1,216 min
Mp.: 210-212° C.

¹H-NMR (500 MHz, d₆-DMSO) δ 11.95 (br s, 1 H), 8.55 (d, 1 H, J=2 Hz), 8.51 (d, 1H, J=2 Hz), 7.45 (s, 1H), 4.33 (t, 1H, J=5.2 Hz), 3.42 (t, 2H, J=6.5 Hz), 2.71 (t, 2H, J=7.5 Hz) 1.67 (m, 2H), 1.48 (m, 2H)

400 mg 3-(4-hydroxy-butyl)-1H-pyrrolo[2,3-b]pyridine-5-carbonitrile were dissolved in 20 mL THF and 0.7 g di-tert-butyldicarbonate and 0.5 mL triethyl amine were added. The reaction mixture was stirred for 72 h at RT. After the usual extraction procedure the crude product 5-cyano-3-(4-hydroxy-butyl)-pyrrolo[2,3-b]pyridine-1-carboxylic acid tert-butyl ester
HPLC: Rt: 3,600 min
HPLC-MS: Rt: 2,000 min
   was directly treated 0.3 mL methanesulfonylchloride and 0.8 mL triethyl amine in 50 mL dichloromethane at 0° C. and for 4 h at RT. After the usual extraction procedure the crude product 5-cyano-3-(4-methanesulfonyloxy-butyl)-pyrrolo[2,3-b]pyridine-1-carboxylic acid tert-butyl ester
HPLC: Rt: 4,190 min
HPLC-MS: Rt: 2,100 min
   was directly transferred to the next step.

0.4 g 4-(2,3-dihydro-benz[1,4]dioxin-6-yloxy)-piperidine were dissolved in 10 mL DMF and added to a suspension of 0.14 g sodium hydride in 5 mL DMF. After 30 min a solution of 0.4 g crude 5-cyano-3-(4-methanesulfonyloxy-butyl)-pyrrolo[2,3-b]pyridine-1-carboxylic acid tert-butyl ester in 5 mL DMF was added. The reaction mixture was heated for 12 h. After the usual extraction the crude product was dissolved in 10 mL acetone and aqueous HCl was added until pH 3. The solution was evaporated and the crude product was re-crystallized from ether, giving 10 mg 3-{4-[4-(2,3-Dihydro-benzo[1,4]dioxin-6-yloxy)-piperidin-1-yl]-butyl}-1H-pyrrolo[2,3-b]pyridine-5-carbonitrile as amorphous solid.
HPLC: Rt: 4,320 min
HPLC-MS: Rt: 1,915 min
Mp: 240-242° C.

¹H-NMR (500 MHz, d₆-DMSO) δ 12.15 (br s, 1 H), 9.45 (br s, 1 H), 8.57 (dd, 2H, J=2 Hz, J=5.6 Hz), 7.52 (s, 1H), 6.78 (t, 1H, J=9 Hz), 6.54 (m, 2H), 4.21 (dd, 4H, J=2 Hz, J=15 Hz), 3.15 (m, 8H), 1.88 (m, 9H).

### Example 4

### Results From Receptor Binding Experiments

Serotonin re-uptake inhibition was determined by means of synaptosomal re-uptake inhibition as described by Perovic & Muller et al. (Arzneim-Forsch. Drug. Res. 1995, 45:1145-1148) with [³H]5-HT as tracer and imipramine as reference.

Norepinephrine uptake was determined as described in the Perovic & Muller work with [³H]NE as tracer and protriptyline as reference.

Dopamine uptake inhibition was determined as described by Janowsky et al. (Neurochem. 1986, 46:1272-1276) with [³H]DA as tracer and GBR12909 as reference.

[³H]Dopamine uptake inhibition is as provided below:

Tissue preparation—A crude synaptosomal membrane preparation from various regions of rat brain was prepared as described by Berger et al. (Eur. J. Pharmacol. 1985, 107:289-290). Brain tissue from adult male Sprague-Dawley rats (150-200 g) was homogenized in 10 volumes of ice-cold sucrose (0.32 M) using a Teflon glass homogenizer, and centrifuged at 1000 g for 10 min. The supernatant was centrifuged at 23000 g for 20 min and the resulting pellet was resuspended in 200 volumes of 50 mM Tris-HCl buffer (pH 7.7) containing 120 mM NaCl at a setting of 5 for 20 seconds. The final protein concentration was 100-200 μg protein/mL, as determined by the method of Lowry et al. (J. Biol. Chem. 1951, 193:265-275).

The uptake of [³H]dopamine in a crude synaptosomal preparation was measured as described by Harris et al. (Life Sci. 1973, 13:303-312). Measurements were carried out in Beckman Biovials containing 400 μL of the membrane preparation, 100 μL of buffer or drugs, 100 μL of [³H]dopamine and 1.4 mL of buffer (50 mM Tris-HCl, pH 7.7, containing 120 mM NaCl and 0.01% bovine serum albumin) in a total volume of 2.0 mL. Tubes were incubated at 25° C. for 45 min, and the incubations were terminated by rapid vacuum filtration over Whatman GF/B filters. The filters were rinsed three times with 4 mL of ice-cold buffer (Tris-HCl, 50 mM, pH 7.7, containing 120 mM NaCl) and the radioactivity remaining on the filters was measured by conventional liquid scintillation spectrometry. Specific binding, defined as the difference in binding observed in the presence and absence of azindole (5 μM), was approximately 70-80% of the total binding at a ligand concentration of 1 nM.

Assay tubes contained 250 μL of the tissue preparation, 50 μL of [³H]dopamine (40 nM) 200 μL of buffer or various uptake inhibitors, and 1.0 mL of Krebs-Ringer bicarbonate buffer (pH 7.4). The tubes were incubated for 2 min at 37° C. and rapidly filtered aver Whatman GF/C glass filters. The filters were rinsed three times with 4 mL of ice cold Krebs Ringer bicarbonate buffer. Identically prepared tubes were incubated on ice for determination of non-specific uptake. Radioactivity remaining on the filters was measured by conventional liquid scintillation spectrometry.

Binding properties at the 5-HT_{1A} receptor were determined by means of the 5-HT_{1A} binding assay as described in the literature by Mulheron et al. (J. Biol. Chem. 1994, 269:12954-12962) with [³H]8-OH-DPAT as ligand and 8-OH-DPAT as reference.

TABLE 1

| Example | Neurotransmitter | IC₅₀ | 5-HT_{1A} |
|---|---|---|---|
| No. | Receptor | (nmol/L) | Kᵢ Binding |
| 1 | Serotonin reuptake | ++ | |
| 1 | 5-HT_{1A} | ++++ | +++ |
| 1 | Norepinephrine | +++ | |
| 1 | Dopamine | +++++ | |
| 2 | Serotonin reuptake | +++++ | |
| 2 | 5-HT_{1A} | ++ | + |
| 2 | Norepinephrine | ++++ | |
| 2 | Dopamine | +++++ | |

Legend for IC₅₀ and Kᵢ binding: + = 1-50 nmol/L; ++ = 51-100 nmol/L; +++ = 101-150 nmol/L; ++++ = 151-200 nmol/L; +++++ = >200 nmol/L.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit and scope of the specification and appended claims.

1. A compound comprising a general structural Formula (I) 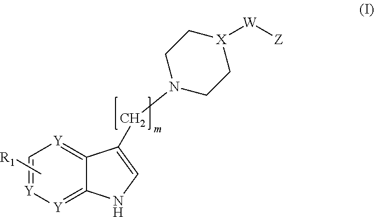
wherein
X is N or CH;
Y each independently is N or CH;
W is (CH₂)ₙ, O, S or N;
R₁ is OH, OA, CN, halo, COR₃, CH₂R₃, or SO₂R₃;
R₃ is OH, OA, NH₂, NHA, or NA₂;
A is C₁₋₆ alkyl that optionally may be substituted or unsubstituted;
Z is a 3-12 membered, unsaturated or unsaturated, mono- or polycyclic ring optionally having one to four heteroatoms and that may be substituted or unsubstituted;
m is 2-6;
n is 0-4;
or a racemic mixture, enantiomer, diastereoisomer, physiologically acceptable salt, solvate, hydrate, solution or prodrug thereof.

2. The compound of claim 1 wherein the optional substituent is selected from the group consisting of OH, OA, NH₂, NHA, NA₂, COR₃, monocyclic or polycyclic carbocycle, a monocyclic or polycyclic heterocycle, or a solvate, hydrate, or prodrug thereof.

3. The compound of claim 1 wherein R₁ is a mono-substituent selected that is cyano, OH, NH₂, halo, or OA, or a physiologically acceptable salt, solvate, hydrate, or prodrug thereof.

4. The compound of claim 1 wherein aryl is benzofuranyl or benzodioxinyl, each of which optionally may be substituted or unsubstituted, or a racemic mixture, enantiomer, diastereoisomer, physiologically acceptable salt, solvate, hydrate, solution or prodrug thereof.

5. The compound of claim 4 wherein the optional substituent is COR₃, or an enantiomer, diastereoisomer, physiologically acceptable salt, solvate, hydrate, or prodrug thereof.

6. The compound of claim 1 selected from the group consisting of:
a. 5-{4-[4-(5-cyano-1H-pyrrolo[2,3-b]pyridin-3-yl)-butyl]-piperazin-1-yl}-benzofuran-2-carboxylic acid amide;
b. 5-{4-[4-(5-cyano-1H-pyrrolo[3,2-b]pyridin-3-yl)-butyl]-piperazin-1-yl}-benzofuran-2-carboxylic acid amide; and
c. 3-{4-[4-(2,3-dihydro-benzo[1,4]dioxin-6-yloxy)-piperidin-1-yl]-butyl}-1H-pyrrolo[2,3-b]pyridine-5-carbonitrile,
or a racemic mixture, enantiomer, diastereoisomer, physiologically acceptable salt, solvate, hydrate, solution or prodrug thereof.

7. A pharmaceutical composition comprising a compound of claim 1 or a racemic mixture, enantiomer, diastereoisomer, physiologically acceptable salt, solvate, hydrate, solution or prodrug thereof, a physiologically acceptable carrier, excipient or diluent and, optionally, at least one further active pharmaceutical ingredient.

8. A process for preparing a compound of claim 1 comprising the steps of:
a. Reacting an amino-cyano-pyridine with iodine in the presence of an acetate to provide a salt and an aqueous phase;
b. extracting the aqueous phase with solvent to provide an organic layer;
c. drying, evaporating and purifying the organic layer to produce amino-iodo-cyano-pyridine crystals; and
d. reacting the amino-iodo-cyano-pyridine crystals with a substituted alkyne in the presence of a suitable base, an adduct, and lithium chloride to produce a desired, substituted azaindole.

9. The compound of claim 1, or a racemic mixture, enantiomer, diastereoisomer, physiologically acceptable salt, solvate, hydrate, solution or prodrug thereof, as a medicament.

10. A method for treating a patient suffering from a neurologic disorder, comprising administering to said patient an effective amount of a compound according to claim 1, or a racemic mixture, enantiomer, diastereoisomer, physiologically acceptable salt, solvate, hydrate, solution or prodrug thereof.

11. The method according to claim 10, or a racemic mixture, enantiomer, diastereoisomer, physiologically acceptable salt, solvate, hydrate, solution or prodrug thereof, wherein the neurologic disorder comprises a 5-HT and/or HT_{1A} receptor disorder.

12. The method according to claim 10, or a racemic mixture, enantiomer, diastereoisomer, physiologically acceptable salt, solvate, hydrate, solution or prodrug thereof, wherein the neurologic disorder is anxiety, depression, schizophrenia, hypertension, Lewy body dementia, Tourette's Syndrome, Huntington's Chorea, Alzheimer's disease, stroke, dementia, dyskinesia, Parkinson's disease, psychosis, neurosis, or a neurodegenerative disease.

13. The method according to claim 12, or a racemic mixture, enantiomer, diastereoisomer, physiologically acceptable salt, solvate, hydrate, solution or prodrug thereof, wherein the neurologic disorder is anxiety, depression, hypertension, or a neurodegenerative disease.

14. A kit comprising separate packs of
(a) a therapeutically effective amount of a pharmaceutical composition according to claim 7, and
(b) a therapeutically effective amount of a pharmaceutical composition comprising a further active pharmaceutical ingredient.
